(19) United States
(12) Patent Application Publication
(10) Pub. No.: US 2021/0393890 A1
(43) Pub. Date: Dec. 23, 2021

(54) Pain Reducing Anesthetic Cooling Device

(71) Applicants: William Lynders, Guilford, CT (US); Michael John Lynders, Shelton, CT (US)

(72) Inventors: William Lynders, Guilford, CT (US); Michael John Lynders, Shelton, CT (US)

(21) Appl. No.: 17/347,746

(22) Filed: Jun. 15, 2021

Related U.S. Application Data
(60) Provisional application No. 63/040,108, filed on Jun. 17, 2020.

(51) Int. Cl. A61M 5/42

(52) U.S. Cl. CPC A61M 5/422; A61M 2021/0022; A61M 2205/3606; A61M 2205/59

(57) ABSTRACT

The pain reducing anesthetic cooling device of the invention reduces pain and minimizes the anxiety often associated with needle injection procedures in both children and adults. The invention reduces pain by precisely anesthetizing a targeted area in a manner that prevents cooling excessive areas of the skin, targets the injection site with a temporary skin impression as a guide for needle injection placement, and makes it easy for medical staff, home care givers or the patient to use and store the device. Additional features of the invention include removable toys, spinning elements and sound and light features to distract and engage the patient. Additionally, the cooling solution may contain colorants such as dyes and particulate additives such as glitter to increase the visual appeal of the device.

### CROSS REFERENCE TO RELATED APPLICATIONS

This application claims benefit to provisional application 63/040,108 filed on Jun. 17, 2020.

### TECHNICAL FIELD

The present invention relates to a pain reducing device, and more particularly to a pain reducing device that uses a cold portion to anesthetize a target area of the body.

### BACKGROUND

Fear of needles is often associated with pain and anxiety in both pediatric and adult patients. These negative associations often begin at an early age with childhood vaccinations and continue throughout adulthood. Several techniques are used to mitigate this pain and anxiety but are used infrequently because they have limitations which do not make them feasible for use in routine vaccinations and other injection procedures. They are particularly difficult to use during large scale vaccination efforts such as COVID-19 and influenza vaccination administration. Currently, the most common practice to alleviate fear and anxiety in a pediatric patient is having the adult accompanying the child hold the child while distracting them. This technique has limitations as it does not anesthetize the target area for the needle procedure so the child will still feel discomfort which then leads to associating needle injections with pain, which contributes to the cycle of needle anxiety and fear. Another option, for both pediatric and adult patients, requires the application of topical numbing agents available in various creams, sprays or patches. This option is expensive and requires a long period of time for the target area to be anesthetized for it to be a practical solution in a clinic setting or large-scale vaccination events. Another option is the use of vapocoolant sprays. These sprays comprise gases that are flammable and are irritants to the eyes. The spray anesthetizes any area of the body that is exposed and comes into contact with the spray, thus it fails to precisely target the area to receive the needle procedure. Additionally, these sprays may cause occupational exposure issues and are toxic to the environment. Another device attempts to reduce pain by combining vibration and ice. This device is expensive and is designed for multi-use applications resulting in infection control complexity and thus is not suited for large scale vaccination events.

Thus there is a need for a pain reduction device suitable and adaptable to pediatric and adult subjects that is inexpensive, single-use, fast acting, safe, easily stored in large quantities and precisely targets the area for the needle injection or procedure, without any of the above listed or other disadvantages.

### SUMMARY OF THE INVENTION

The present invention is drawn to an apparatus for facilitating needle procedures in pediatric and adult subjects, it is more specifically directed to reduce pain by anesthetizing a targeted area of the body for patients receiving vaccines and other medical procedures requiring a needle, without deleteriously affecting body surfaces outside of the localized area. The device is useful in hospital, clinic and home settings. It is easily used by healthcare professionals, self-administering patients or a non-professional care giver. This is particularly useful in settings providing large scale vaccinations such as COVID-19 vaccines, influenza vaccines and other vaccines.

The invention is drawn to reducing pain and anxiety for children or adults prior, during and post injections. Reducing pain and anxiety is instrumental in vaccine and other medical procedures to ensure compliance. It has been reported in the literature such as Relevant Statistics: McLenon, J. “The fear of needles: A systemic review and meta-analysis” Journal of Advanced Nursing, 2019, vol 75, Issue 1, pp. 30-42 that 20-50% of adolescents have needle fear and anxiety, 20-30% of young adults have needle fear and anxiety, 16% of adults avoid flu vaccination because of needle fear, 50% of women with pregnancy-induced diabetes fear self-injections with a needle, 40% of caregivers for diabetic children fear injecting their child and 65% of dialysis patients try to avoid injecting themselves. These statistics demonstrate that fear of needles is a significant contributing factor to the lack of vaccine compliance and other medical procedures as well as avoiding self-administration procedures, pain abortive infusions, fertility treatments and other treatments, and contributes to a patient's avoidance of often necessary diagnostic testing and therapeutic procedures. The search for better ways to improve the experience of vaccinations or other medical injections becomes even more critical for diseases like Covid-19 or other pandemic diseases which require large scale vaccinations and treatments.

The present invention is based on an understanding of human physiology and of the laws of thermodynamics. When an injection is administered the needle injures the tissue as it penetrates the injection location, the nerves in the area respond by sending a pain signal to the brain. Injections are administered into three types of tissue: the skin, subcutaneous and intramuscular. The human skin has far more nerve endings than the subcutaneous or intramuscular locations which results in a sharp and focal pain response. The subcutaneous or intramuscular injections, by contrast, produce a duller, vague ache from a needle puncture. A theory posited by research suggests that one type of nerve transmission can block another type of sensation at the spinal cord where nerves merge in a hub-like manner. Effective counter pain sensations may include pressure, cold and vibration applied to an injection site. Cooling of the nerve tissue is particularly effective to reduce pain. It has been found that cooling of nerve tissue to only 50 degrees Fahrenheit reduces conduction of nerve signals by a third. Warm blood circulating back to the cooled tissue however reduces some of the benefits of cooling the injection site. The present invention mitigates the warming of the injection site and enhances the pain reduction provided by cooling of the targeted area for an injection by also applying pressure to a small circle of nerves affected by the needle. The pressure compresses superficial blood vessels trying to rewarm the area; the cold and pressure sensations may block the pain signals at the spinal cord thus enhancing pain reduction. Further, the structure of the device around the ice allows the person applying the pressure to not feel the cold of the ice and the device insulates the ice from the environmental warmth.

Another benefit of the present invention is post-injection when the re-application of the device to the area having received the injection continues to impart pain reducing benefits. The application of the pain reducing cooling device and pressure minimizes the deeper pain associated with the muscle and reduces bleeding from injured vessels which contribute to pain after an injection. Another embodiment of the present invention that enhances pain reduction, especially in pediatric patients is distraction. Distraction is known to block pain transmission at higher levels of the nervous system such as in the brain stem and the cortex. An embodiment of the present invention includes a toy which amongst other features may spin, rotate, reveal a surprise to engage and distract the child while an injection is being administered. Other features may include sound or light.

The present invention utilizes principles of thermodynamics to reduce pain. An embodiment of the invention uses the concepts and properties of ice and its melting point. It is a basic scientific tenet that adding certain compounds to water causes it to freeze at lower temperature. For example, an ice cube comprising essentially of water is warmer than an ice cube comprising water and salt additives. The extra cooling stored in the solid phase of ice is another important principal. Cool gel packs traditionally store less cooling power because the additive gelling agent does not allow for an ice melting phase, resulting in a gel pack that warms up faster than a non-gel containing pack. An embodiment of the present invention specifically maximizes thermodynamic properties of materials for maximum cooling of the targeted area. Even more targeted cooling is achieved by the shape of the coolant containing structure. For example, a flat disc shaped ice pack has an ice phase, but it loses cooling ability at an unacceptable rate due to the high surface to volume ratio. Thus, the present invention uses water solutions containing an additive to reach a freezing temperature below 32 degrees Fahrenheit. The additive is preferably a salt such as sodium chloride. The additive may also be other non-toxic additives known in the art. The solution may contain other additives known in the art that do not affect the freezing phase of the solution, for example additives that may prevent leakage. As the ice feature in the ice phase pulls heat from the body it will remain at a temperature below 32 degrees Fahrenheit for a period of time to cool the injection site to receive the injection and it is still sufficiently cold to be applied to the injection site post injection to continue to cool the skin and muscle to reduce pain. The coolant cartridges are preferably a cylinder shape. This shape is the best shape to maximize the surface area to volume ratio to deliver the most beneficial cooling to the targeted body area.

Another embodiment of the present invention is the application of the pain reducing cooling apparatus to provide a cooling numbing device that targets nerve pain conduction associated with minor superficial injuries to the skin from accidents, insect bites, small abrasions, cuts, contusions or burns. The cooling device is suited for home, office, school, and medical facility settings. Its convenient size and design appeal contribute to the timeliness of applying the cooling device to the injury to achieve the desired reduction of pain and swelling. Additionally, the device may be re-cooled and re-applied to the injury as needed.

The pain reducing cooling anesthetic devices of the present invention are not only effective to cool a very specific, targeted area of the body receiving a needle procedure, but they are also useful post procedure, where they may be reapplied to further reduce pain and discomfort from the procedure. The shape and size of the devices are particularly suited for targeted applications, delivering pain reducing benefits without deleteriously affecting surrounding tissue. Although the invention is described with human patients, it is understood that it may be applied to animals requiring targeted pain reduction.

The invention is a pain reducing anesthetic cooling device having a coolant which is applied prior to an injection to cool the targeted area for an anesthetic effect. The device having a cartridge housing and coolant may be two separate parts. The device is particularly suited to provide targeted anesthetic properties without impacting surrounding tissues. The pain reducing cooling is maximized by the shape of the device, where the surface area and volume are designed to impart maximum, rapid cooling of the body area while the coolant retains its freezing temperature for a period of time to make it particularly useful post injection or for a minor injury where it can be applied multiple times.

The invention may further comprise a retaining cap attached to the cartridge housing. The retaining cap may be attached by, e.g., a snap-fit or screw fit. The retaining cap allows for efficient cooling. For example, it may have a ring-shaped cap having an orifice or it may be a ring-shaped cap having a thin layer which allows efficient cooling of the subject's targeted body area. The ring-shaped cap with the orifice is a preferred embodiment as it allows for the coolant containing cartridge to protrude through the orifice for optimal contact with the targeted body area to achieve maximum cooling. The retaining cap may have a flange on its perimeter for snap fitting to the cartridge housing. Alternatively, the retaining cap may snap fit to the cartridge housing with a plurality of tabs spaced apart on its perimeter.

The pain reducing anesthetic cooling device may further comprise an attachment to the cartridge housing and extends therefrom, such as a ring member or a protruding member. A ring member facilitates holding the cooling apparatus to the target area for the necessary time period to cool the skin to reduce pain for the needle injection. Alternatively, a protruding member therefrom can assist in holding the cooling apparatus to the target area. Additionally, the protruding member may be configured to removably attach a toy. The toy may provide a distraction to a child especially during the cooling of the injection site and during the injection. Any toy having a receiving slot to complement the protruding member is suitable to removably attach to the protruding member. Examples of toys include, but are not limited to, action figures, seasonal toys, teddy bears, dolls, promotional merchandise, and all animal shapes. To increase the distraction of a child the toy may also contain movable parts and/or reveal compartments as well as sound and light features. The housing also provides insulation of the cold to the fingers holding the device and the environmental warmth which will melt the ice.

The pain reducing device's retaining cap may be configured to freely spin in either direction. The spinning movement provides distraction to a pediatric patient. The retaining cap which spins may be shaped in any form. Examples of the spinning retaining cap include but are not limited to flowers, stars, badges, dinosaurs, and other animals.

The pain reducing device may further comprise a flexible hinge joining the cartridge housing and retaining cap.

The invention is also particularly useful for preparing the body part receiving multiple needle injections, for example during childhood vaccinations when often multiple injections are given at the same time. The pain reducing device comprises a second cartridge housing and a second coolant, wherein the cartridge housings are attached to each other at an appropriate distance for medical professionals to administer separate vaccines.

The invention is drawn to a method of anesthetizing a targeted area of a body part in preparation for a needle injection. The method comprises applying pressure with a pain reducing cooling device described above for a period of time to cool the targeted area to numb the pain by reducing nerve conduction, then removing the cooling device before the injection. The pain reducing cooling device, when removed from the targeted body area after being held to achieve the necessary cooling, may leave a gentle impression on the skin. The impression may serve as a guide to the person injecting the needle. A particular benefit of an impression on the skin is that it identifies the area that has been cooled where it is most beneficial to perform the needle injection. The center of the impression is the most ideal spot for the needle injection since that is the area of the skin and muscle that is at the lowest temperature and thus imparts the greatest pain reduction. Optionally, the coolant device may be placed on the injection site post-injection to further minimize pain.

Yet another embodiment of the invention is drawn to a method of using the device to reduce swelling and pain resulting from minor injuries, cuts, insect bites, minor burns and abrasions. The method precisely targets the injured area and delivers pain and swelling reducing benefits without engaging the non-injured areas surrounding the injured body portion. The method comprises applying pressure to the injured body portion, with the pain reducing device, for a sufficient amount of time to stem blood flow and reduce pain. The pain reducing device is removed once a desired level of comfort has been reached. The procedure may be repeated until the subject reaches a further acceptable level of comfort and reduction of pain.

The pain reduction cooling device not only reduces pain but minimizes the anxiety often associated with needle injection procedures. The cooling device of the invention reduces pain by precisely anesthetizing a targeted area in a manner that prevents cooling excessive areas of the skin, targets the injection site with a temporary skin impression as a guide for needle injection placement, and makes it easy for medical staff, home care givers or the patient to use and store the device. Additional features of the invention contribute to distracting the patient, in addition to toys, and spinning elements, sound and light features, the coolant may contain colorants such as dyes and particulate additives such as glitter to increase the visual appeal.

The coolant may be a small plastic cartridge containing a frozen substance such as ice comprising water, a solution of water and sodium chloride or other salts, a solution of water and non-toxic additives known in the art to reduce the freezing point of water, or other cold substances which can be stored in a freezer, preferably in sheets of multiple units.

The cartridge shells could be made of vacuum formed polyethylene terephthalate glycol (PETG) sheet material that is transparent. This sheet material may be about 0.015 inch thick. The cartridge shells are easily grouped and filled with cooling material. The refrigerant cooling agent may contain coloring agents or particulates such as glitter for additional appeal and interest. The material could be sealed in place with a heat-sealed foil utilizing standard sealing techniques. The cartridge sheets may be die-cut in a way that would allow individual cartridges to be snapped off as needed. The two-part pain reducing device may be made by the injection molding process using High Impact Styrene or Polypropylene. The choice of materials will be based on the need for the retaining cap to snap over the cartridge housing and be able to spin freely. The retainment cap snap-fit could be designed to allow for the replacement of the cartridge with a fresh cartridge from the refrigerator or designed to be permanently fixed once snapped in place. The retaining cap may contain a plurality of tabs on the outer perimeter, or a continuous flange to snap fit over the cartridge housing. The retaining cap and cartridge housing may also be configured to close as a screw cap.

The embodiments above may be used together in any combination thereof. For example, the cartridge housing, cartridge and retaining cap may be used with an optional ring; or the cartridge housing, cartridge and retaining cap may be used with a protruding member and an optional removable toy; another example is a double cartridge with a protruding member with the optional removable toy; yet another combination is the cartridge housing and cartridge; yet another combination is a cartridge housing, a cartridge, and the spinning retaining cap; yet still another combination is the cartridge housing, cartridge, retaining cap and the flexible connection holding the cartridge housing and the retaining cap together.

### BRIEF DESCRIPTION OF THE DRAWINGS

The present disclosure will be better understood by those skilled in the pertinent art by referencing the accompanying drawings, where like elements are numbered alike in the several figures, in which:

FIG. 1A is a perspective assembly view of one embodiment of the pain reduction device;

FIG. 1B is a cross sectional view of the pain reduction device from FIG. 1A;

FIG. 1C is an exploded perspective view of the pain reduction device from FIG. 1A;

FIG. 1D is a top orthographic view of the pain reduction device from FIG. 1A;

FIG. 1E is an orthographic front view of the pain reduction device from FIG. 1A;

FIG. 1F is a cross sectional front view of the pain reduction device from FIG. 1D;

FIG. 1G is an exploded orthographic view of the pain reduction device from FIG. 1C;

FIG. 1H is a top view of the retaining cap from FIG. 1A;

FIG. 1I is a front view of the retaining cap from FIG. 1H;

FIG. 1J is a side view of the retaining cap from FIG. 1H;

FIG. 1K is a cross section front view of the retaining cap from FIG. 1H;

FIG. 1L is a bottom view of the retaining cap from FIG. 1H;

FIG. 1M is a perspective view of the cartridge of the pain reduction device from FIG. 1C;

FIG. 1N is an exploded perspective view of the cartridge of the pain reduction device from FIG. 1M;

FIG. 10 is a top view of the cartridge of the pain reduction device from FIG. 1M;

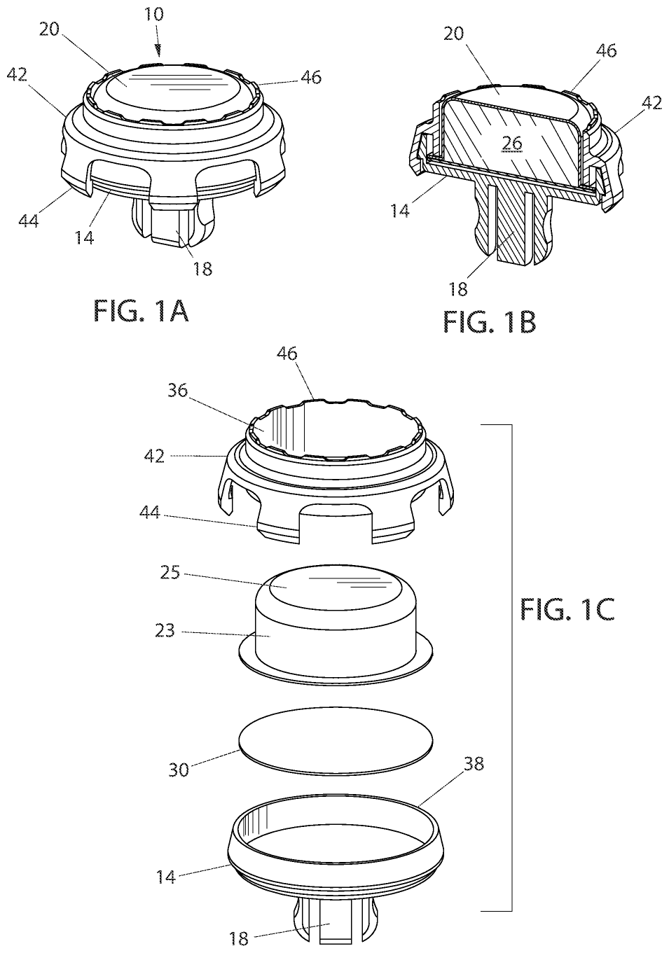

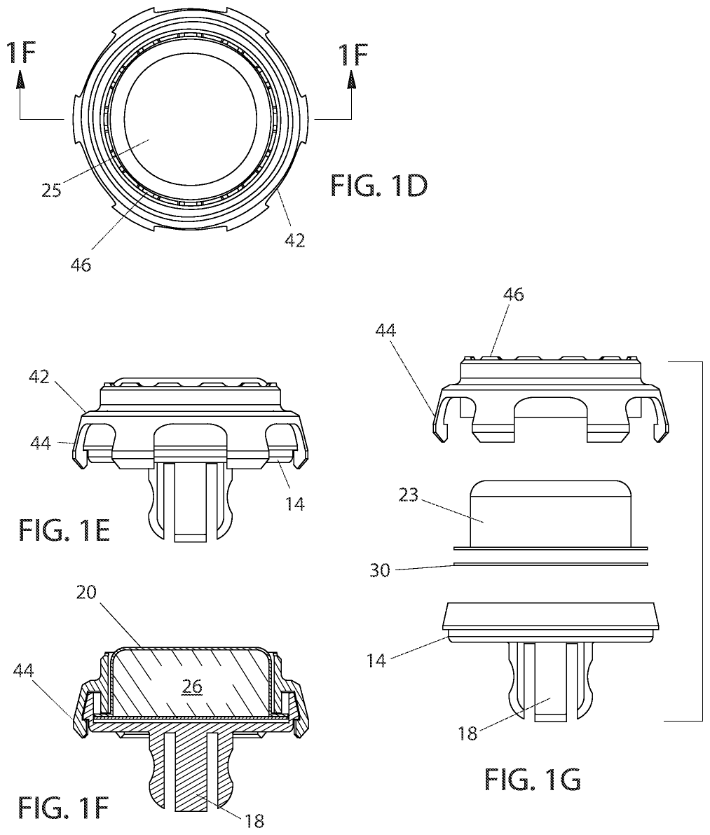

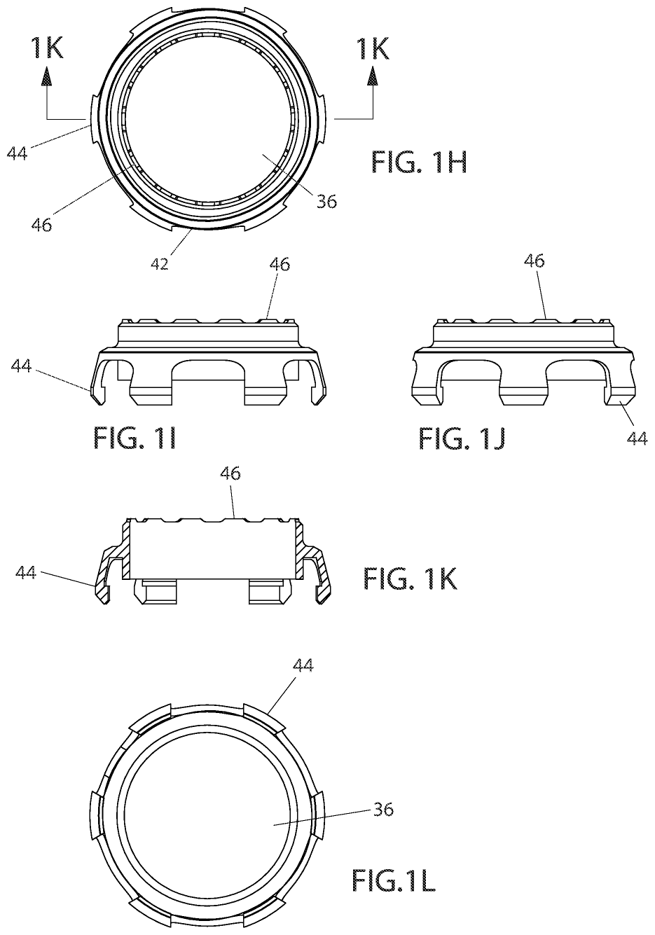

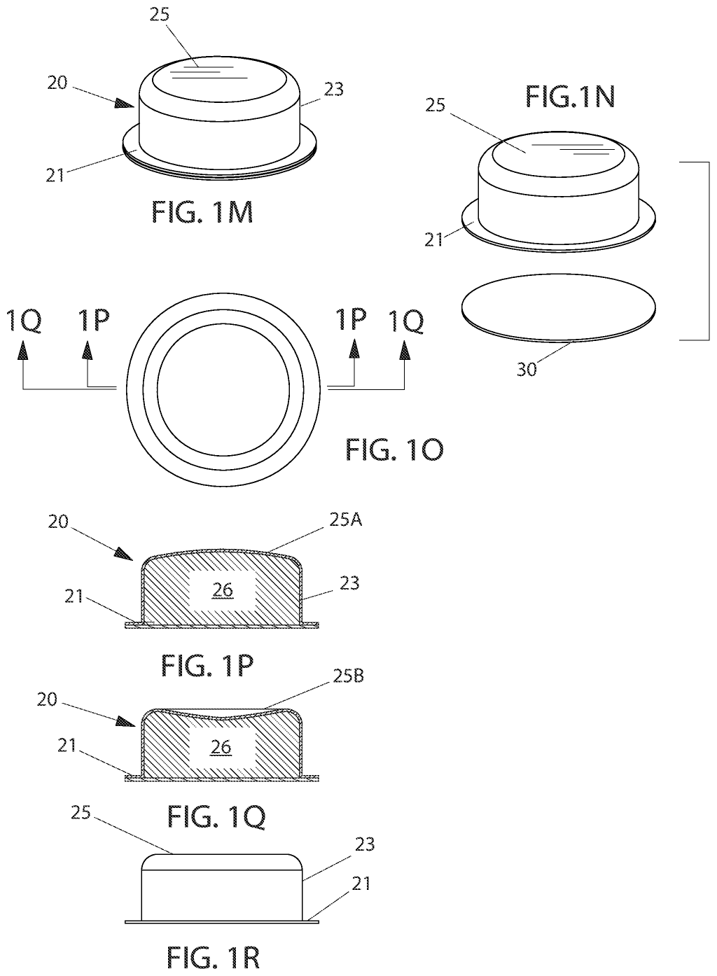

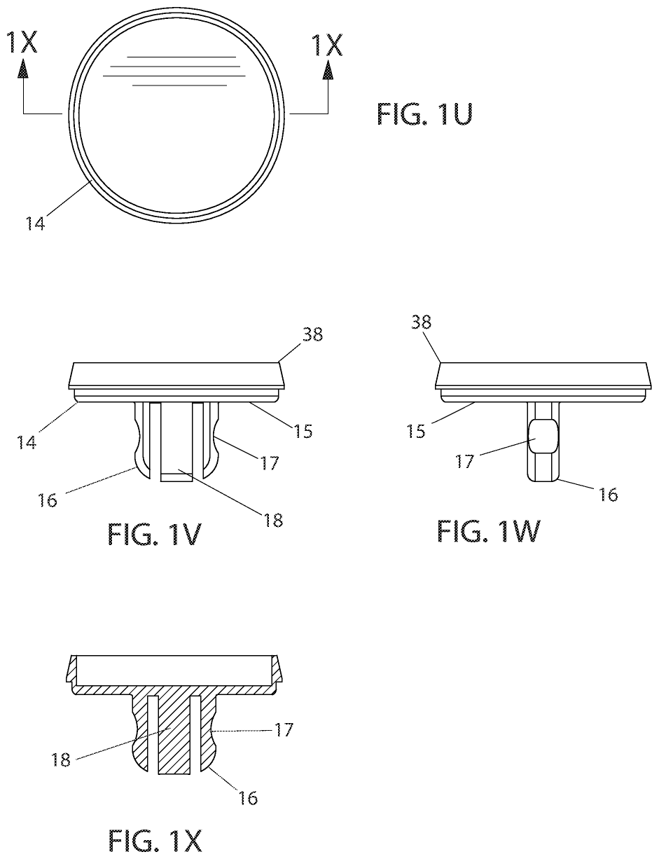

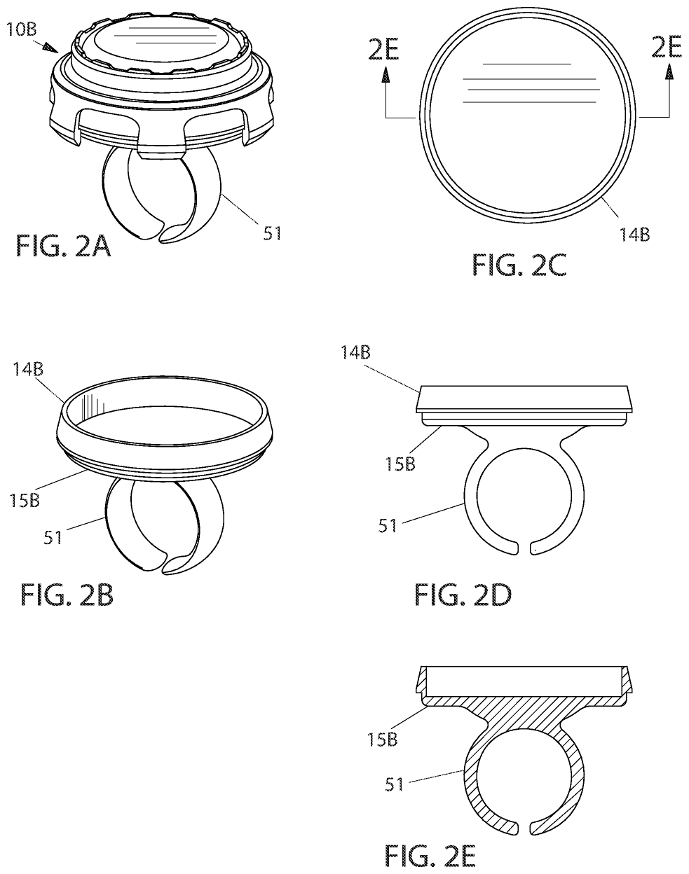

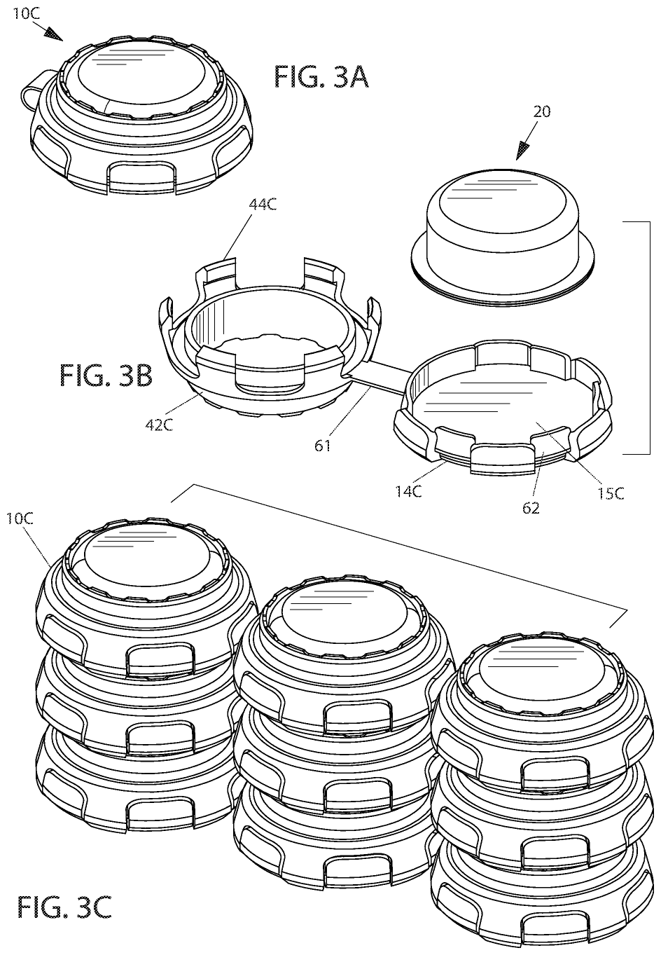

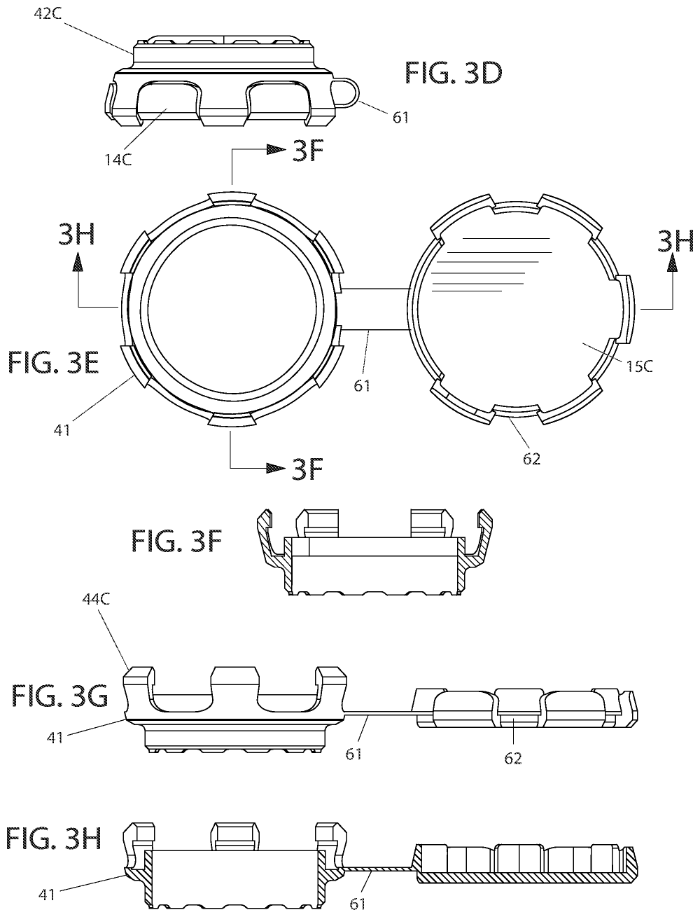

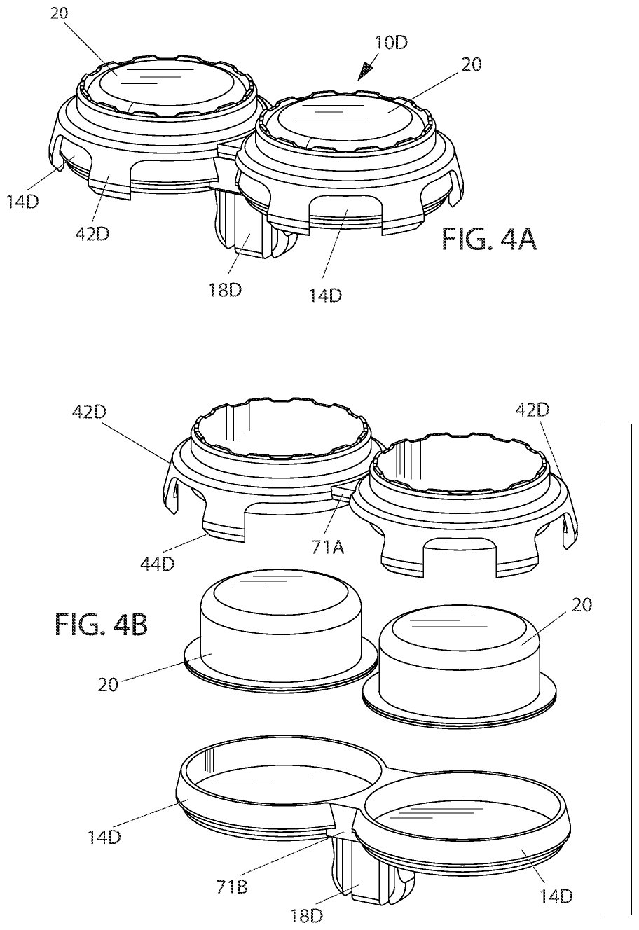

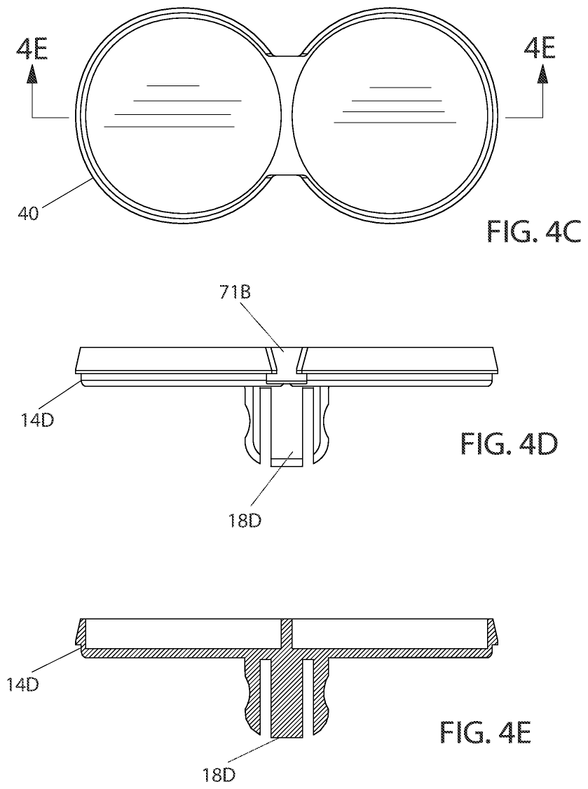

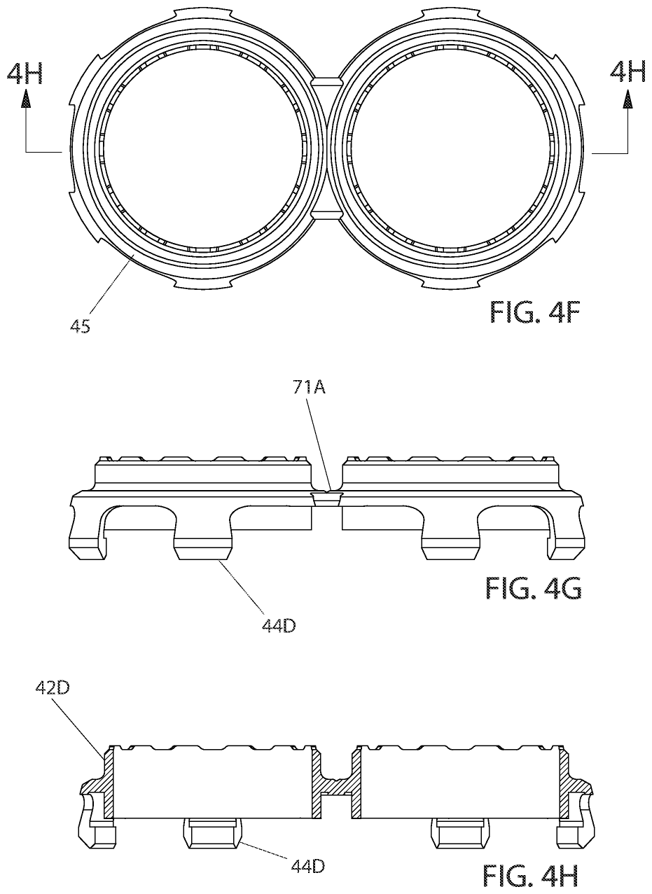

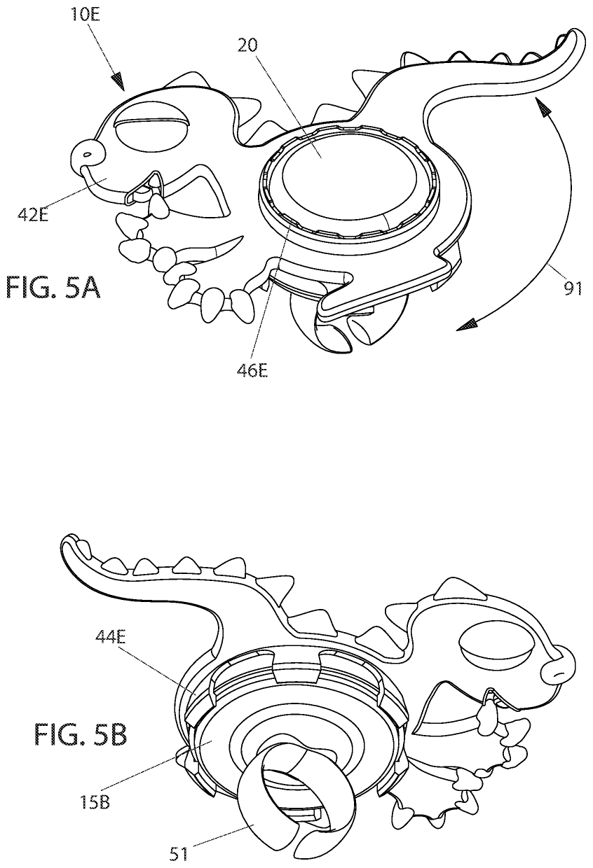

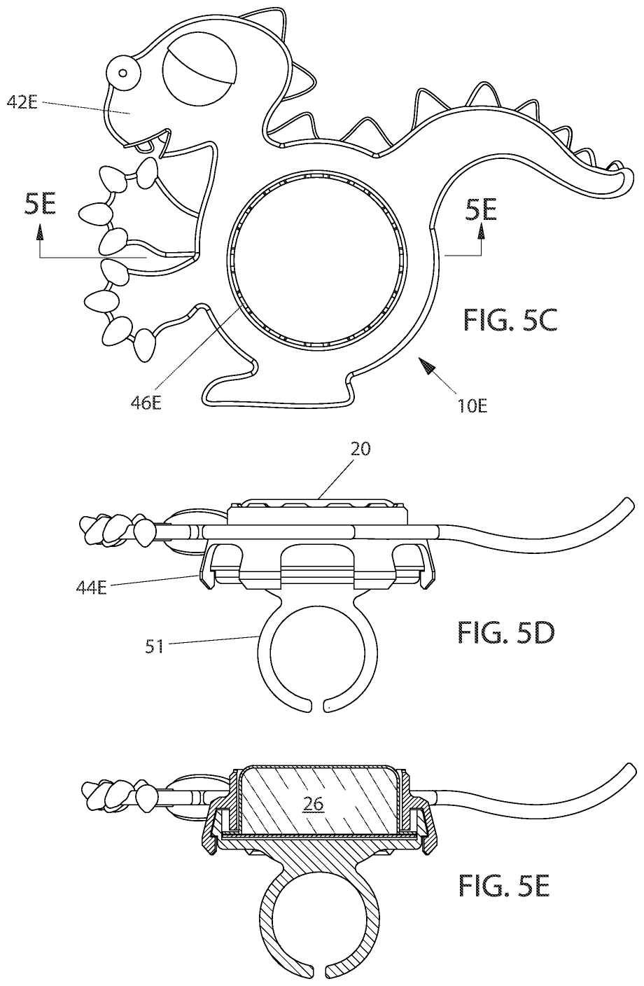

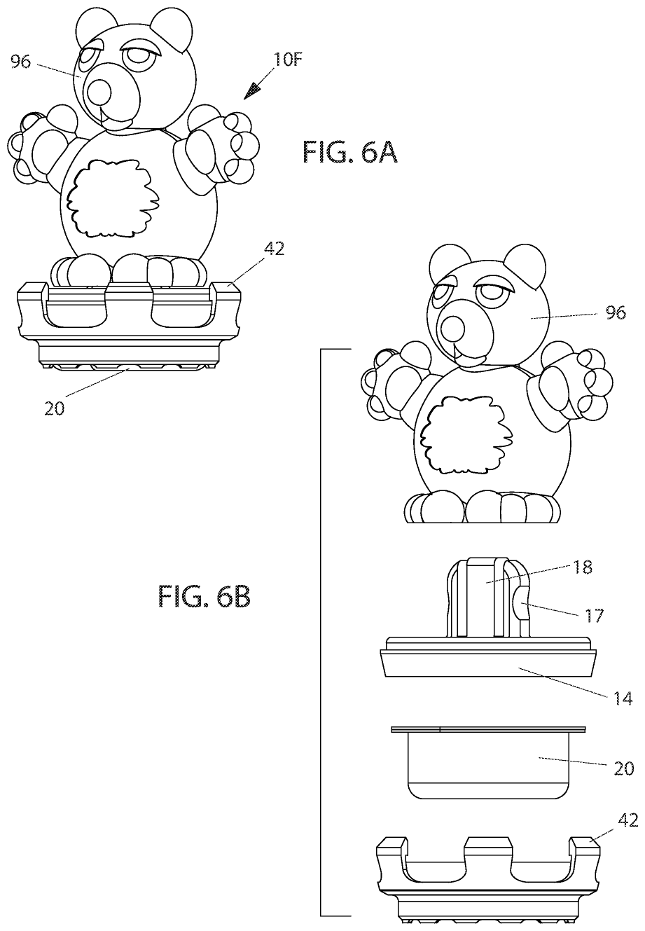

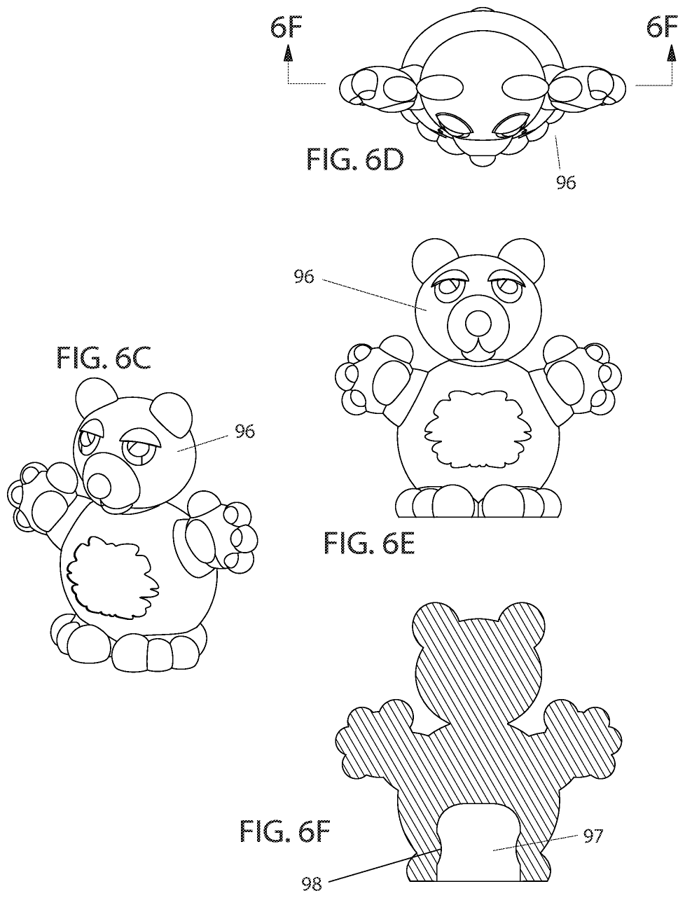

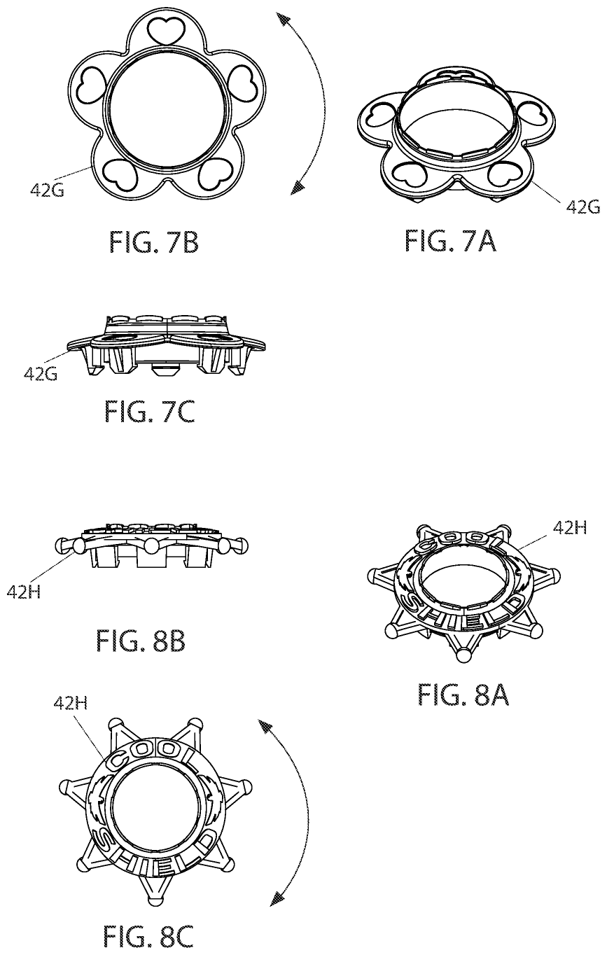

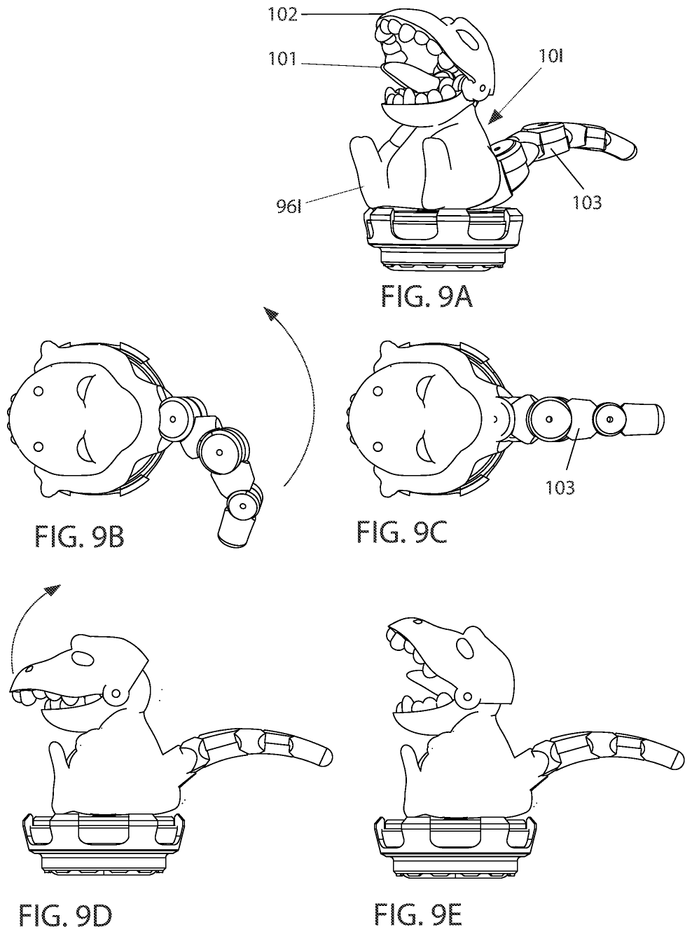

FIG. 1P is a cross sectional view of the coolant cartridge with a heat-sealed foil covering as shown in FIG. 1M when in the frozen stage;

FIG. 1Q is a cross sectional view of the coolant cartridge with a heat-sealed foil covering as shown in FIG. 1M in a non-frozen liquid stage;

FIG. 1R is a front view of the cartridge of a pain reduction device of 1M with coolant in a neutral volume between frozen and liquid volumes;

FIG. 1U is a top view of the cartridge housing of FIG. 1C;

FIG. 1V is a front view of the embodiment of the cartridge housing having a protruding member attached thereto of FIG. 1U;

FIG. 1W is a side view of the embodiment of the cartridge housing having a protruding member attached thereto of FIG. 1U;

FIG. 1X is a cross section view of the embodiment of the cartridge housing of FIG. 1U;

FIG. 2A is a perspective assembly view of another embodiment of the pain reducing device having a ring handle;

FIGS. 2B is a perspective view of a cartridge housing with the ring handle feature of FIG. 2A;

FIGS. 2C is a top view of the cartridge housing having the ring handle feature of FIG. 2B;

FIGS. 2D is a front view of the cartridge housing with a ring handle feature of FIG. 2B;

FIGS. 2E is a cross section view of the embodiment of the cartridge housing with a ring handle feature of FIG. 2C;

FIGS. 3A shows a perspective view of the pain reducing device assembly having a flexible hinge and no handle on the cartridge housing;

FIG. 3B is an open exploded perspective view of the pain reducing device with a flexible hinge and a cartridge of FIG. 3A;

FIG. 3C is a perspective view of multiple flat pain reducing devices with a flexible hinges and cartridges demonstrating stacking for ease of storage;

FIG. 3D is an orthographic front view of the pain reducing device assembly with a flexible hinge and cartridge of FIG. 3A;

FIG. 3E is a top view of the pain reducing device with a flexible hinge in an open position without the cooling cartridge;

FIG. 3F is a side cross sectional view of the retaining cap of the pain reducing device with a flexible hinge of FIG. 3E;

FIG. 3G is a front view of the open position of the pain reducing device with a flexible hinge without the cooling cartridge;

FIG. 3H is a front cross-sectional view of the open position of the pain reducing device with a flexible hinge of FIG. 3E;

FIG. 4A is the pain reducing device having double-cartridges adjacent to one another and a protruding member bridging the adjacent cartridges;

FIG. 4B is an exploded perspective view of the double cartridge pain reducing device of FIG. 4A;

FIG. 4C is a top view of the cartridge housing for the double cartridge pain reducing device of FIG. 4A;

FIG. 4D is a front view of the cartridge housing for the double cartridge pain reducing device of FIG. 4C;

FIG. 4E is cross section view of the cartridge housing for the double cartridge pain reducing device of FIG. 4C;

FIG. 4F is a top view of the retaining cap for the double cartridge pain reducing device of FIG. 4A;

FIG. 4G is a front view of the retaining cap for the double cartridge pain reducing device of FIG. 4F;

FIG. 4H is a cross sectional front view for the retaining cap of the double cartridge pain reducing device of FIG. 4F;

FIG. 5A is a perspective assembly view of the pain reducing device demonstrating a spinning retaining cap configured as a toy, the toy is exemplified as a dinosaur;

FIG. 5B is a perspective bottom view of the pain reducing device of FIG. 5A;

FIG. 5C is a top view of the pain reducing device of FIG. 5A;

FIG. 5D is a front view assembly of the pain reducing device FIG. 5C;

FIG. 5E is a cross sectional view of the pain reducing device of FIG. 5C;

FIG. 6A is an embodiment of the invention having a pain reducing device with a detachable toy;

FIG. 6B is an exploded view of the pain reducing device of FIG. 6A;

FIG. 6C is a perspective view of the pain reducing device with detachable toy of FIG. 6B;

FIG. 6D is a top view of the pain reducing device with detachable toy of FIG. 6C;

FIG. 6E if a front view of the pain reducing device detachable toy of FIG. 6D;

FIG. 6F is a cross sectional front view of the pain reducing device with detachable toy of FIG. 6D;

FIG. 7A is a flower shaped embodiment of the invention from the perspective view of the spinning retaining cap for the pain reducing device;

FIG. 7B is a top view of the spinning retaining cap for the pain reducing device of FIG. 7A;

FIG. 7C is a front view of the spinning retaining cap for the pain reducing device of FIG. 7A;

FIG. 8A is a badge shaped embodiment of the invention from the perspective view of the spinning retaining cap for the pain reducing device;

FIG. 8B is a front view of the pain reducing device of FIG. 8A

FIG. 8C is a top view of the pain reducing device demonstrating the spinning retaining cap shaped as a badge of FIG. 8A;

FIG. 9A is an embodiment of the pain reducing device having a detachable toy wherein the toy has moving parts including moving jaw and tongue and articulating tail;

FIG. 9B is a top view of the pain reducing device of FIG. 9A showing movement of the articulating tail;

FIG. 9C is a top view of the pain reducing device of FIG. 9A;

FIG. 9D is a side view of the pain reducing device of FIG. 9A showing the movement of the jaw;

FIG. 9E is a side view of the pain reducing device of FIG. 9A showing the jaw and tongue in the open position;

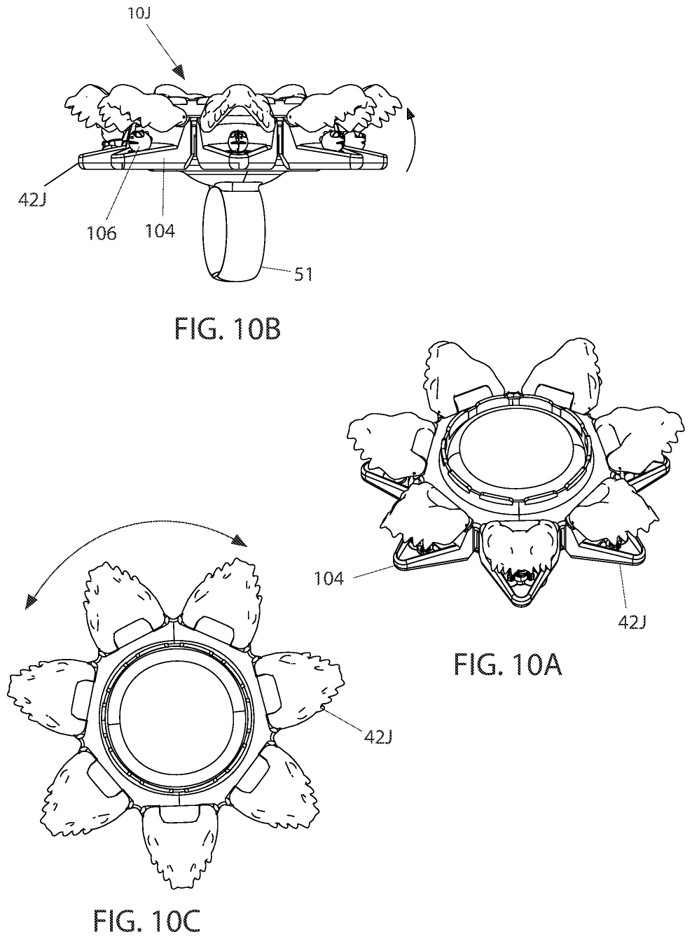

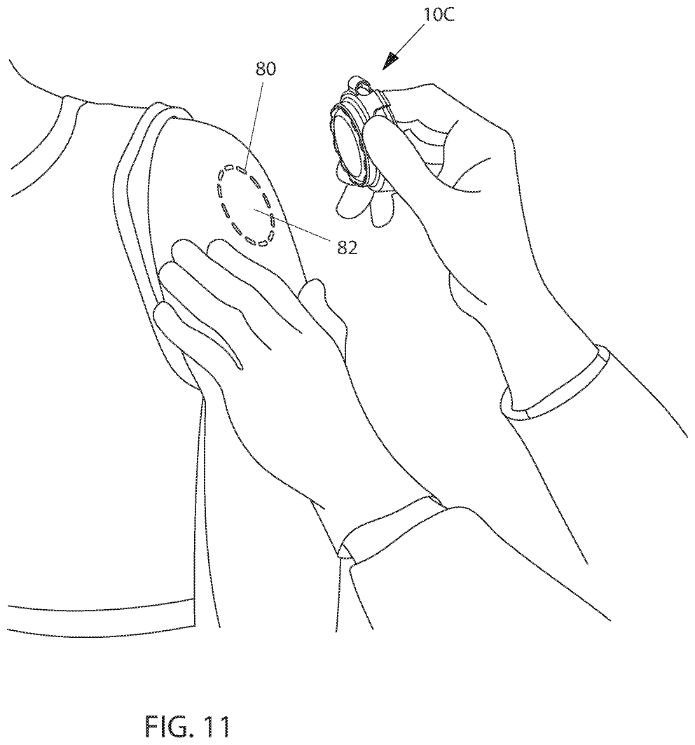

FIG. 10A is an embodiment of the pain reducing device having a spinning retaining cap with multiple compartments that swing open to reveal another toy or feature;

FIG. 10B is a side view of the pain reducing device of FIG. 10A that shows the up and down motion of the jaws;

FIG. 10C is a top view of the pain reducing device of FIG. 10A that shows spinning;

FIG. 11 is the representation of a pain reducing device after being applied to a targeted area on the arm of a patient.

### DETAILED DESCRIPTION OF THE INVENTION

The invention is best described by referring to the figures. FIGS. 1A, 1B and 1C show one embodiment of the pain reduction device 10. The device may comprise a cartridge housing 14, with a protruding member 18 extending therefrom. The protruding member is configured to receive a removable toy 96 as seen in FIG. 6B, by snap fitting into a complimentary receiving slot 97 in the toy as seen in FIG. 6F. The protruding member also serves as a holder when applying pressure to the body area receiving a needle injection. A cartridge 20, having a cartridge side wall 23, cartridge top 25 and a cartridge lid 30, sits in the cartridge housing 14, within the cartridge housing wall 38. The cartridge contains a coolant solution therein. The cartridge lid 30 may be a foil and may be heat sealed to the cartridge via known methods in the art. A retaining cap 42 attaches by snap fit to the cartridge housing 14 with the tabs 44. The retaining cap 42 is shown with an orifice 36 that allows the cartridge to contact the body area requiring anesthetizing. The retaining cap 42 may have raised projections 46 surrounding the orifice 36 which precisely delineate the body area cooled by the pain reducing device 10. An impression 80 on the skin as seen in FIG. 11 is made by raised projections on the pain reduction device 10C. Shown in FIG. 11, 80 is a circular impression; however, other shapes may provide the same function. The impression 80 will be made in similar manner with all the pain reduction devices of the instant invention of FIGS. 1A, 2A, 4A, 5A, 6A, 7A, 8A, 9A and 10A. Moreover, the area of the skin where the cartridge has cooled the skin may have a flushed or reddened hue after application of the pain reducing device to guide the person administering the needle injection. The impression 80 in FIG. 11 further guides the person injecting the needle by directing them to place the needle in the center 82 of the area surrounded by impression 80 as that is the coldest part of the surface area that has been cooled thus imparting the most beneficial pain reducing properties.

FIGS. 1D-1G further demonstrate the pain reducing device of FIG. 1A. FIG. 1D shows the top of the cartridge 25 that will contact the targeted body area to achieve anesthetic pain reducing cooling of the surface surrounded by the projections 46 on the retaining cap 42 that will leave an indentation of the skin after pressure is applied to achieve the cooling to reduce pain during an injection. The retaining cap 42 and cartridge housing 14 form the two-part cartridge housing of this embodiment. They may be made by an injection molding process using High Impact Styrene or Polypropylene. The choice of materials will be based on the need for the retaining cap 42 to snap over the cartridge housing 14 enclosing the cartridge. The retaining cap snap-fit could be designed to allow for the replacement of the cartridge with a fresh cartridge from the freezer or designed to be permanently fixed once snapped in place. The retaining cap is shown as having a plurality of projections 46 on the outer perimeter of the orifice 36, however, the projections 46 may be replaced by a single solid element surrounding the perimeter of the orifice thus the impression on the skin will be a continuous line rather than an impression with spaces in the perimeter of the impression as seen in FIG. 11. The embodiment of FIGS. 1A-1K demonstrates a snap fitting retaining cap 42 over cartridge housing 14 using tabs 44. FIG. 1L shows the bottom of the retaining cap 42 with the orifice 36 and tabs on the cap for snap fitting. However, the retaining cap may also be configured to screw fit over the cartridge housing 14. Additionally, the tabs 44 of the retaining cap 42 may be substituted by a single flange configuration, wherein the retaining cap snaps onto the cartridge housing with the continuous flange rather than the plurality of tabs 44. FIG. 1F shows the coolant solution 26 in the cartridge 20.

FIGS. 1M-1R show the cartridge 20, having a rim 21 at the bottom of the column-shaped cartridge wall 23 and a top portion 25. The cartridge contains a cooling solution 26 as seen in 1P and 1Q. The solution comprises coolants such as water, a water solution containing a temperature reducing additive such as sodium chloride or other salts and non-toxic additives known in the art. The cartridge is filled with a liquid state solution 26 as seen in FIG. 1Q where the top portion of the cartridge is concave 25B which when frozen will expand as seen in FIG. 1P. The expansion of the solution as it freezes causes the top portion of the cartridge 25A to be convex as seen in FIG. 1P. The cartridge shells could be made of vacuum formed (should we say what this stands for) PETG sheet material that is transparent. This sheet material may be about 0.015 inch thick and allows for expansion of the coolant during freezing. The convex surface of the top of the cartridge aides in cooling the target area of the body. FIG. 1N shows cartridge lid 30 configured to fit on the rim 21. Materials suited for the lid include material that can be heat-sealed, with known heat-sealing techniques in the art, such materials include foils or any material suited for heat sealing the lid 30 to the rim 21 of the cartridge 20. The cartridges filled with refrigerant coolant and sealed could be stacked in appropriate groupings for ease of storage. The refrigerant cooling agent may contain coloring agents or particulates such as glitter for additional appeal and interest.

FIGS. 1U, 1V, 1W and 1X demonstrate the cartridge housing 14. The cartridge housing 14 contains a bottom 15 and a cartridge housing wall 38 perpendicular to the bottom 15. The cartridge housing is configured to fit the cartridge shell of FIGS. 1P-1R. Attached to the outside wall of the bottom portion 15 of the cartridge housing is a protruding member 18. Protruding member 18 may be a three-prong protrusion 16 having a notch 17 on the protrusions to receive a removable toy having a slot 97 as seen in FIG. 6F or other receiving mechanism to secure the removable toy to the protruding member 18 of FIGS. 1V, 1W and 1X.

FIG. 2A is a pain reducing device 10B where the protruding member 18 is replaced with a ring member 51. The ring member is attached to the bottom 15B of the cartridge housing 14B. The cartridge housing is configured to fit the retaining cap 42 and cartridge 20 of FIGS. 1P-1R. The ring 51 facilitates holding the device while applying pressure to a targeted area of the body to allow for cooling of the needle injection site.

FIGS. 3A-3H show the pain reducing device 10C comprising a cartridge housing 14C connected to the retaining cap 42C by a flexible hinge 61 and cartridge 20. The flexible hinge 61 allows the retaining cap 42C to snap fit over the cartridge 20 once it has been placed into the cartridge housing 14C. The cartridge housing 14C has a plurality of offset indent sections 62 offset from each other on the perimeter of the cartridge housing bottom 15C. The retaining cap 42C complementary snap fit over the cartridge housing offset sections 62 with tabs 44C to snap close the retaining cap and cartridge housing. FIG. 3C shows a plurality of pain reducing devices 10C of this embodiment stacked and ready for use. FIG. 3D is a side view of the pain reducing device 10C showing the flexible hinge 61 in a closed position. FIG. 3E shows the top view of an open position of the hinged retaining cap and cartridge housing 41 for the pain reducing device 10C having the tabs 44C which snap fit into offset sections 62 of the cartridge housing. FIGS. 3F-3H show 41 in side view.

FIGS. 4A-4H show the pain reducing device 10D, particularly useful for pediatric patients receiving multiple vaccinations at one time, in the same limb, usually about one to one and a half inches apart. In FIGS. 4A and 4B the pain reduction device 10D can be seen with two cartridges 20, two retaining caps 42D and two cartridge housings 14D. A double retaining cap attachment post 71A connects two retaining caps. A double cartridge housing attachment post 71B connects two cartridge housings. This configuration allows two targeted areas to be anesthetized simultaneously. A single protruding member 18D may be attached to the post connecting the two pain reducing devices. The protruding member 18D is configured to receive a removable toy as seen in FIG. 6B, by snap fitting into a complimentary receiving slot in the toy. The toy may be removed and handed to the child while the pain reducing device is being applied and while the injection occurs. A ring member, not shown, may be used in place of the protruding member. FIGS. 4C-4E show the double cartridge housing 40. FIGS. 4F-4H show the double retaining cap 45.

FIGS. 5A-5E is the pain reducing device 10E having a spinning retaining cap 42E shaped as a figure that spins about the cartridge housing in either direction shown by the arrow 91. The spinning element depicted in the figures is a dinosaur, but any other figure or representation is appropriate to provide the additional distraction especially to a pediatric patient, examples are seen in FIGS. 7A-7C which depict the spinning retaining cap 42G as flower shaped and FIGS. 8A-8C which depict a star shaped badge spinning retaining cap 42H. Another example of the spinning retaining cap 42J is seen in FIGS. 10B-10C, wherein the spinning retaining cap comprises a toy with opening compartments 104 that reveal a part of the toy 106 when opened. The ring 51 facilitates application while applying pressure to the targeted body area.

FIGS. 6A-6F demonstrate the pain reducing device 10F with a toy attachment 96. The cartridge housing 14 with the protruding member 18 attached thereto is configured to receive a toy attachment 96. In FIG. 6B the protruding member 18 has notches 17 to receive a toy with a complimentary slot 97 and raised areas 98 seen in FIG. 6F for clicking into the protruding member opening 17. The toy 96 is shown as a teddy bear, however any figurine, dolls, action figures, entertainment merchandise, seasonal toys, animals or other depictions may be used. Examples are seen in FIGS. 9A-9E where the pain reducing device 101 has a toy attachment 961 which is configured into a dinosaur having movable parts such as the movable jaw 102 and tongue 101 and an articulating tail 103. All figurines or toys may be interchangeable with the same pain reducing device cartridge 20, cartridge housing 14 and retaining cap 42. Multiple figurines may be stored and used for different ages

The disclosed pain reduction device has many advantages. It will anesthetize a precise targeted area on a patients ‘body in preparation for an injection without the need for drugs. The pain reduction device has a toy such as a figurine, or a spinning element, that will keep a child occupied and distracted from the injection. For an adult the device can be made without a toy. One embodiment of the pain reduction device will prepare the patient for two injections on the same limb. The pain reduction device is inexpensive and disposable for proper infection control. However, the pain reduction device may be reusable if the family wants to take it home to use on wounds or insect bites. The toy on the pain reduction device may be replaced with other toys or figurines to appeal to a wide variety of children. The device is designed to be used and stored in a medical clinic in order to give greater access to patients, yet not disrupt medical staff workflow. The small size of the cartridge makes it easy to store many units in a freezer. The small size of the cartridge also minimizes the area cooled on the skin, minimizing the feeling of the cold cartridge on surrounding skin area making vaccinations more comfortable and in turn increasing vaccination compliance rates.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. A pain reducing anesthetic cooling device targeting a body portion of a human or animal comprising:
a cartridge housing and a coolant for cooling the targeted area to a temperature for reducing pain nerve conduction, wherein the volume to surface area ratio of the device maximizes the localized cooling of the isolated area.

2. A pain reducing anesthetic cooling device targeting a body portion of a human or animal according to claim 1 comprising:
the cartridge housing and the coolant being two separate parts.

3. A pain reducing anesthetic cooling device targeting a body portion of a human or animal according to claim 1 further comprising a retaining cap;
said coolant comprising a cartridge positioned within the cartridge housing; and said retaining cap being attached to the cartridge housing.

4. A pain reducing anesthetic cooling device targeting a body portion of a human or animal according to claim 1 further comprising:
an attachment on the cartridge housing, said attachment is selected from the group consisting of a ring and a protruding member.

5. A pain reducing anesthetic cooling device targeting a body portion of a human or animal according to claim 4 wherein said attachment is a protruding member.

6. A pain reducing anesthetic cooling device targeting a body portion of a human or animal according to claim 5, further comprising:
a removable toy attachment having an opening for snapfitting onto the protruding member.

7. A pain reducing anesthetic cooling device targeting a body portion of a human or animal according to claim 6 wherein said removable toy is selected from the group consisting of a teddy bear, doll, action figure, seasonal figurines, entertainment figurines, entertainment merchandise and animal shapes.

8. A pain reducing anesthetic cooling device targeting a body portion of a human or animal according to claim 3 further comprising a feature selected from the group consisting of light and sound.

9. A pain reducing anesthetic cooling device targeting a body portion of a human or animal according to claim 1 wherein the coolant is selected from the group consisting of water and water solution containing a temperature reducing additive.

10. A pain reducing anesthetic cooling device targeting a body portion of a human or animal according to claim 1 wherein the coolant contains additives selected from the group consisting of coloring agents and coloring particulates.

11. A pain reducing anesthetic cooling device targeting a body portion of a human or an animal according to claim 3 wherein the retaining cap has a flange on its perimeter for snap fitting to the cartridge housing.

12. A pain reducing anesthetic cooling device targeting a body portion of a human or an animal according to claim 3 wherein the retaining cap has a plurality of tabs spaced on its perimeter for snap fitting to the cartridge housing.

13. A pain reducing anesthetic cooling device for targeting a body portion of a human or an animal according to claim 3 wherein the retaining cap comprises a spinning retaining cap that spins around the cartridge housing.

14. A pain reducing anesthetic cooling device for targeting a body portion of a human or an animal according to claim 13 wherein the spinning retaining cap shape is selected from the group consisting of a dinosaur, flower, star, badge and animals.

15. A pain reducing anesthetic cooling device targeting a body portion according to claim 3 wherein the cartridge housing and retaining cap are attached via a flexible hinge.

16. A pain reducing anesthetic cooling device targeting a body portion according to claim 15 wherein the cartridge housing having a plurality of offset indents and the retaining cap having a plurality of tabs to complementary snap fit into one another.

17. A pain reducing anesthetic cooling device targeting a body portion according to claim 1 further comprising:
a second cartridge housing and a second coolant attached to the first cartridge housing and first coolant.

18. A pain reducing anesthetic cooling device targeting a body portion according to claim 17 further comprising:
a post affixed between the first and second cartridge housings,
a first retaining cap, a second retaining cap for snap fitting over the first and second cartridge housings and a protruding member, wherein said protruding member is attached to the first and second cartridge housings.

19. A pain reducing anesthetic cooling device targeting a body portion according to 18 further comprising a removable toy attachment said removable toy having an opening for snap-fitting onto the protruding member.

20. A pain reducing anesthetic cooling device targeting a body portion according to claim 19 wherein said removable toy is selected from the group consisting of a teddy bear, doll, action figure, seasonal figurines, entertainment figurines, entertainment merchandise and animal shapes.

21. A method for anesthetizing a targeted body portion of a human or animal to reduce pain in needle injection procedures comprising applying pressure on the human part body with a pain reducing anesthetic cooling device wherein the cooling device comprises a cartridge housing and a coolant for cooling the targeted area to a temperature for reducing pain nerve conduction comprising: cooling the targeted body portion; removing the cooling device; performing the needle injection and optionally reapplying the cooling device post procedure.

22. A method for anesthetizing a targeted body portion to reduce pain from an injury, insect bite, burn or abrasion, comprising applying pressure on the affected body part with a pain reducing anesthetic cooling device wherein the cooling device comprises a cartridge housing and a coolant for cooling the targeted area to a temperature for reducing pain nerve conduction to the affected body area; applying pressure to the affected body area with the device; cooling the targeted body area until the pain reduces and removing the cooling device and optionally reapplying the cooling device until the pain further diminishes.
